(19) United States
(12) Patent Application Publication
(10) Pub. No.: US 2003/0043884 A1
(43) Pub. Date: Mar. 6, 2003

(54) Probe cover of ear thermometer

(76) Inventors: Kuan Yuan Ko, Chubei City (TW); Yunn Nan Li, Chubei City (TW); Chung Shin Wu, Chubei City (TW); Tao Cheng Lu, Chubei City (TW); Lu Ts Liu, Chubei City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 09/940,452

(22) Filed: Aug. 29, 2001

(51) Int. Cl. G01K 1/08; A61B 5/00; G01J 5/02

(52) U.S. Cl. 374/158; 600/474; 600/549

(57) ABSTRACT

The present invention provides a probe cover of ear thermometer comprising a ring, a thin film, and a paper film. The thin film can be penetrated by infrared rays. The paper film is disposed on the surface of an open end of the thin film. The paper film and the open end of the thin film are fixed on the ring by means of hot-pressing to form a cover body, which extends upwards. The present invention has the advantages of simple structure and simplified manufacturing process, hence decreasing the manufacturing time and reducing the cost of material.

### FIELD OF THE INVENTION

The present invention relates to a cover and, more particularly, to a probe cover covered on an ear thermometer.

### BACKGROUND OF THE INVENTION

Medical thermometers are indispensable tools during a general diagnosis process. Among all body portions used to measure the body temperature, the temperature of the eardrum can much more represent the temperature inside the body than the mouth cavity, the rectum, or the armpit. An infrared ear thermometer utilizes infrared radiation emitted from the eardrum in the ear canal to measure the temperature of the eardrum. When measuring infrared radiation in the ear canal, the front section of a probe of the ear thermometer needs to penetrate deeply into the outer ear canal. In order to prevent contamination and infection of disease when using an ear thermometer to measure the ear temperatures of different patients, a probe cover is sleeved onto the probe of the ear thermometer. The probe cover is discarded once used.

As shown in FIGS. 1 and 2, a prior art probe cover comprises a base 12 with an opening (not shown) formed at the center thereof. A retaining portion 16 is annularly disposed at the periphery of the opening. An open end of a thin film 18 is disposed on the surface of the base 12 at the periphery of the opening, and extends upwards to form a cover body. An annular frame 20 is sleeved downwards from above the cover body to let the periphery of the annular frame 20 be retained on the retaining portion 16, thereby clamping and fixing the open end of the thin film 18 on the base 12.

The design of clamping the thin film 18 on the base 12 through the annular frame 20 needs to first manufacture the base 12 and the annular frame 20, and then join them together. The manufacture is more cumbersome, and the cost is higher. Moreover, if either the base 12 or the annular frame 20 is not satisfactorily manufactured so that they cannot be tightly clamped together, the thin film 18 of the probe cover 10 will easily fall off, resulting in inconvenience in use. There is another kind of probe cover integrally formed of material that can be penetrated by infrared rays. However, for this kind of probe cover manufactured by pressing and deforming material, the quality control in the manufacturing process is more difficult.

Accordingly, in consideration of the above problems, the present invention aims to propose a probe cover formed by means of hot-pressing to fix a thin film on a ring, thereby resolving the problems in the prior art.

### SUMMARY OF THE INVENTION

The primary object of the present invention is to propose a probe cover of simple structure and simplified manufacturing process to decrease the manufacturing time and reduce the cost of material.

A probe cover of the present invention comprises a circular ring with an annular protuberance formed at the periphery of a ring hole thereof. A thin film that can be penetrated by infrared rays is provided. A paper film covers on an open end of the thin film. The paper film and the open end of the thin film are simultaneously fixed on a side face of the annular protuberance and the surface of the circular ring by means of hot-pressing to form a cover body, which extends upwards and toward the ring hole.

The various objects and advantages of the present invention will be more readily understood from the following detailed description when read in conjunction with the appended drawing, in which:

### BRIEF DESCRIPTION OF THE DRAWINGS

FIG. 1 is a structure diagram of a prior art probe cover;

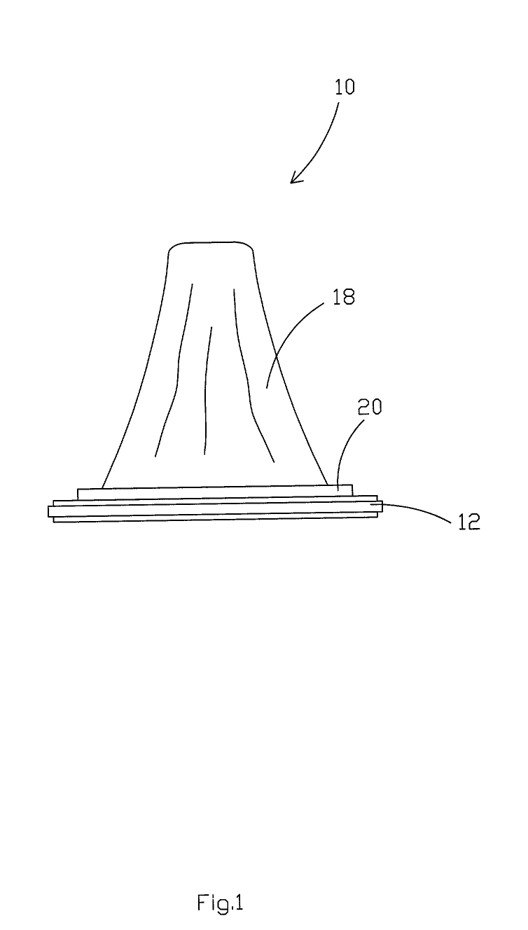

FIG. 2 is an exploded view of a prior art probe cover; and

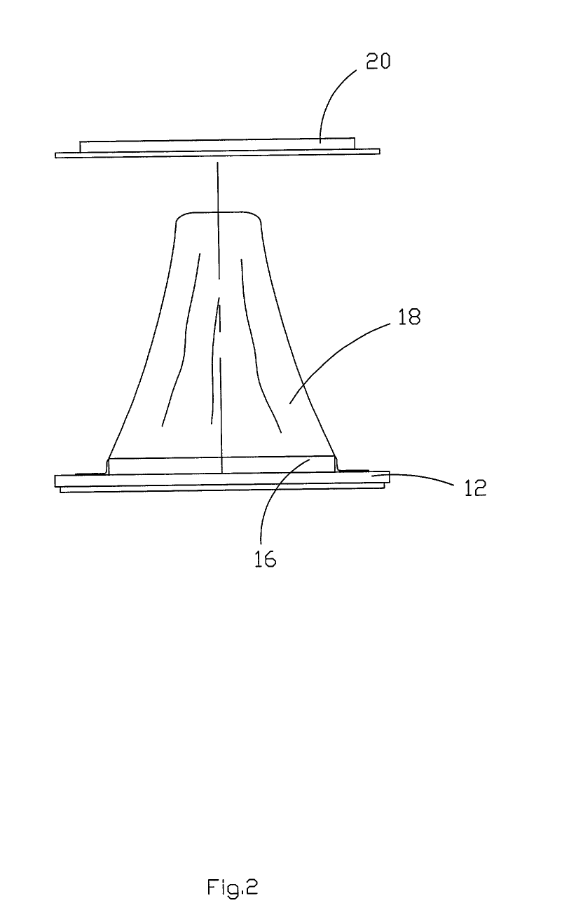

FIG. 3 shows a structure diagram and a partly enlarged view of the present invention.

### DETAILED DESCRIPTION OF THE PREFERRED EMBODIMENTS

The present invention is mainly characterized in that a thin film is fixed on a ring to form a cover body by means of hot-pressing, thereby achieving the advantage of simple manufacture.

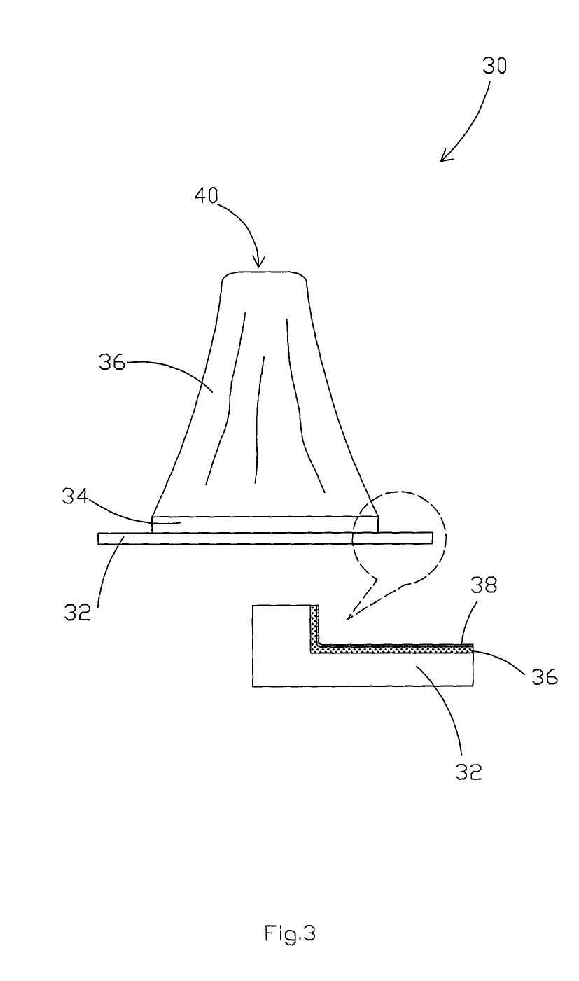

As shown in FIG. 3, a probe cover 30 of the present invention comprises a circular ring 32 with a ring hole at the center thereof. An annular protuberance 34 is formed at the periphery of the ring hole. A thin film 36 that can be penetrated by infrared rays is provided. An open end of the thin film 36 is flatly pasted on a side face of the annular protuberance 34 and the surface of the circular ring 32. A ring-shaped paper film 38 covers on the surface of the open end of the thin film 36. The open end of the thin film 36 and the paper film 38 are fixedly disposed on the circular ring 32 by means of hot-pressing to let the thin film 36 form a cover body, which extends upwards and toward the ring hole. The cover body is used to cover a probe of an ear thermometer.

The circular ring 32 is made of plastic. The ring hole of the ring 32 can dovetail a rear end of the probe of the ear thermometer so that the probe cover can cover on the ear thermometer by retaining the circular ring 32 at the rear end of the probe. A closed end of the thin film 36 forms a window 40 of infrared rays. A probe of an ear thermometer covered by the probe cover probes deeply into the outer ear canal to measure infrared radiation in the ear canal for obtaining the temperature inside the body. The probe cover is discarded once used.

In the present invention, because the thin film and the circular ring are integrally formed by means of hot-pressing, the structure is simpler and the manufacturing process is simplified, hence saving an assembly procedure of clamping the thin film. Moreover, because the paper film covers on the surface of the thin film, in addition that the thin film can be protected so that it will not deform due to high temperature when performing hot-pressing, the joining strength between the thin film and the circular ring can be enhanced to prevent the thin film from falling off.

Although the present invention has been described with reference to the preferred to embodiment thereof, it will be understood that the invention is not limited to the details thereof. Various substitutions and modifications have been suggested in the foregoing description, and other will occur to those of ordinary skill in the art. Therefore, all such substitutions and modifications are intended to be embraced within the scope of the invention as defined in the appended claims.

I claim:

1. A probe cover of ear thermometer covered on a probe of an ear thermometer, comprising:
a ring with a ring hole at a center thereof;
a thin film, an open end thereof being fixedly disposed on said ring and extending toward said ring hole to form a cover body for covering on said probe of said ear thermometer; and
a paper film situated on a surface of said open end of said thin film, said paper film and said open end of said thin film being fixed on said ring by means of hot-pressing.

2. The probe cover of ear thermometer as claimed in claim 1, wherein said thin film can be highly penetrated by infrared rays.
